(19) United States
(12) Patent Application Publication
(10) Pub. No.: US 2024/0019145 A1
(43) Pub. Date: Jan. 18, 2024

(54) AIR CLEANING SYSTEM

(71) Applicant: PATHOGEN REDUCTION SYSTEMS LIMITED, Horley, Surrey (GB)

(72) Inventors: Ian Sinclair, Horley, Surrey (GB); Julia Martin, Horley, Surrey (GB); Robert Mccullough, Sausalito, CA (US)

(21) Appl. No.: 18/254,646

(22) PCT Filed: Nov. 26, 2021
(86) PCT No.: PCT/GB2021/053091

§ 371 Date: May 26, 2023

(30) Foreign Application Priority Data
Nov. 27, 2020 (GB) 2018738.1

(51) Int. Cl. F24F 8/22; F24F 3/16; F24F 13/02

(52) U.S. Cl. CPC F24F 8/22; F24F 3/16; F24F 13/029

(57) ABSTRACT

The present disclosure relates to an air cleaning system comprising: a ducting section comprising an inlet and an outlet; at least one source of ultraviolet radiation arranged to emit ultraviolet radiation into an interior volume of the ducting section, the interior volume between the inlet and the outlet; and a reflective surface arranged to reflect ultraviolet radiation emitted by the source of ultraviolet radiation within the interior volume of the ducting section, wherein the reflective surface is capable of reflecting at least 60% of incident ultraviolet radiation.

### FIELD

The present disclosure relates to an air cleaning system. In particular, the present disclosure relates to an air cleaning system in which air is cleaned using ultraviolet light.

### BACKGROUND

Air handling systems such as heating, ventilation and air conditioning (HVAC) systems are used to supply air to buildings and other locations. Typically, these air handling systems recirculate recycled air within the building. The recirculation of recycled air can involve recirculation of pathogens, if one or more occupants of the building are infected with a particular disease.

Adaptation of existing systems to use only outside air (i.e. so that no recycled air is recirculated) is not practical. This is because the heating and cooling components of existing air handling systems are designed to operate with a maximum non-recycled air fraction of approximately 30%. Running these existing systems at 100% non-recycled air would result in insufficient heating or cooling of the air supply, causing discomfort for the building's occupants and potentially resulting in an unusable working environment. Modifying these existing systems to increase the heating or cooling capacity of the air supply systems would incur significant cost, and would result in substantially higher running costs for the building.

The need to avoid recirculation of contaminated air has been accentuated by the Covid-19 pandemic. As a result of the pandemic, some building air supply systems are being operated to minimise recirculation of recycled air, at the cost of the comfort of the indoor environment. Although this may be acceptable in the summer months, when the recirculated outdoor air is warmer and lower humidity, it will cause significant disruption when recirculating colder, more humid air during the winter. For example, recirculation of the more humid winter air may cause corrosion of internal components. Another effect of the pandemic is that some buildings are operating at significantly reduced occupancy, whereas others are closed completely. This has led to substantial disruption to businesses that depend on maximising occupancy, such as restaurants, nightclubs, and other indoor venues.

Accordingly, there exists a need for an improved air cleaning system that reduces or eliminates the recirculation of contaminated air.

### SUMMARY

This summary introduces concepts that are described in more detail in the detailed description. It should not be used to identify essential features of the claimed subject matter, nor to limit the scope of the claimed subject matter.

According to a first aspect of the present disclosure, there is provided an air cleaning system comprising: a ducting section comprising an inlet and an outlet; at least one source of ultraviolet radiation arranged to emit ultraviolet radiation into an interior volume of the ducting section, the interior volume between the inlet and the outlet; and a reflective surface arranged to reflect ultraviolet radiation emitted by the source of ultraviolet radiation within the interior volume of the ducting section, wherein the reflective surface is capable of reflecting at least 60% of incident ultraviolet radiation.

The ultraviolet radiation emitted by the source of ultraviolet radiation provides an energy flux density within the interior volume, which kills any pathogens in the air flowing through the interior volume. The use of a reflective surface allows photons emitted by the source of ultraviolet radiation to be reflected back into the interior volume of the ducting section, which increases the energy flux density within the interior volume.

The at least one source of ultraviolet radiation may be provided in a recess so as to mitigate obstruction of airflow through the ducting section. Mitigating obstruction of the airflow allows the air cleaning system to be installed in an existing air handling system without having to re-balance the system to account for any restriction of the airflow through the system.

The reflective surface may comprise a material that is capable of reflecting at least 65% of incident ultraviolet radiation. Preferably, the reflective surface may comprise a material that is capable of reflecting at least 70% of incident ultraviolet radiation. More preferably, the reflective surface may comprise a material that is capable of reflecting at least 75% of incident ultraviolet radiation. More preferably, the reflective surface may comprise a material that is capable of reflecting at least 80% of incident ultraviolet radiation. More preferably, the reflective surface may comprise a material that is capable of reflecting at least 85% of incident ultraviolet radiation. More preferably, the reflective surface may comprise a material that is capable of reflecting at least 90% of incident ultraviolet radiation. More preferably, the reflective surface may comprise a material that is capable of reflecting at least 95% of incident ultraviolet radiation. Reflecting an increased amount of incident ultraviolet radiation further increases the energy flux density within the interior volume.

The material may comprise one or more of: polytetrafluoroethylene, PTFE, nylon, ultra-high-molecular-weight polyethylene, UHMWPE, or any combination of the foregoing materials. Preferably, the material comprises PTFE, which has high reflectivity to ultraviolet radiation.

The air cleaning system may further comprise a removable casing, wherein the source of ultraviolet radiation is disposed within the removable casing. Providing the source of ultraviolet radiation within a removable casing allows the air cleaning system to be maintained. Specifically, providing the source of ultraviolet radiation within a removable casing allows the source of ultraviolet radiation (e.g. one or more ultraviolet lamps) to be replaced when necessary.

The removable casing may further comprise the reflective surface. The removable casing may comprise a back wall and side walls that define an enclosure in which the source of ultraviolet radiation is disposed. The reflective surface may be disposed on the back wall of the removable casing.

The ducting section may comprise at least one wall that defines the interior volume, wherein the removable casing is arranged to cover an opening in the at least one wall. The ducting section may comprise a plurality of walls that define the interior volume, wherein the air cleaning system comprises a plurality of removable casings, each of the plurality of removable casings being arranged to cover an opening in a respective one of the plurality of walls.

Each of the plurality of removable casings may comprise a source of ultraviolet radiation. By providing a source of ultraviolet radiation in each of a plurality of removable casings, the airflow through the internal volume is exposed to ultraviolet radiation from multiple directions, which increases the irradiation that each virus is exposed to.

The at least one source of ultraviolet radiation may comprise a plurality of ultraviolet lamps. The ultraviolet lamps may be mercury lamps. The ultraviolet lamps may be amalgam lamps. The ultraviolet lamps may be LEDs. The at least one source of ultraviolet radiation may comprise an excimer lamp or excimer plate.

Adjacent ones of the plurality of ultraviolet lamps may be spaced apart from one another to provide a gap between the adjacent ones of the plurality of ultraviolet lamps. A portion of the reflective surface may be exposed to the ultraviolet radiation through the gap between the adjacent ones of the plurality of ultraviolet lamps. Providing a gap between adjacent lamps allows a portion of the reflective surface to be exposed to the ultraviolet radiation. This means that a greater portion of the ultraviolet radiation is reflected, thereby increasing the energy flux density within the interior volume.

The total area of the gaps may be between about 50% and about 80% of an area of a surface on which the ultraviolet lamps are disposed. The total area of the gaps may be between about 70% and about 80% of the area of the surface on which the ultraviolet lamps are disposed. The total area of the gaps may be between about 75% and about 80% of the area of the surface on which the ultraviolet lamps are disposed. Increasing the total area of the gaps increases the amount of reflected ultraviolet radiation and therefore maximises the energy flux density within the interior volume.

Each of the plurality of ultraviolet lamps may comprise a longitudinal axis, wherein the longitudinal axis of each of the plurality of lamps is parallel to a flow path from the inlet to the outlet. This maximises the time that each virus particle is exposed to the ultraviolet radiation from the lamps.

The at least one source of ultraviolet radiation may comprise a plurality of sources of ultraviolet radiation, wherein the plurality of sources of ultraviolet radiation are arranged to emit ultraviolet radiation into the interior volume from at least two directions. This means that the airflow through the internal volume is exposed to ultraviolet radiation from multiple directions, which increases the irradiation that each virus is exposed to and reduces the likelihood of radiation being blocked by larger particles.

The at least one source of ultraviolet radiation may be arranged to emit ultraviolet-C, UVC, radiation. The wavelength of the UVC radiation may be between about 200 nm and about 280 nm. Preferably, the wavelength of the UVC radiation is between 210 nm and 260 nm. More preferably, the wavelength of the UVC radiation is about 222 nm or about 254 nm. 222 nm and 254 nm radiation have been shown to be effective at killing pathogens. In addition, 254 nm ultraviolet lamps are widely available, therefore providing a cost-effective solution.

According to a second aspect of the present disclosure, there is provided a method of designing an air cleaning system, comprising: determining an energy flux density required to reduce a number of pathogens in a volume of air within a duct of an air handling system, wherein the energy flux density is determined based on the size of the duct and the velocity of airflow through the duct; determining an electrical power of at least one source of ultraviolet radiation required to provide the determined energy flux density; and designing an air cleaning system comprising the at least one source of ultraviolet radiation having the determined electrical power.

The method allows an electrical power requirement to be determined for an air handling system in order to effectively destroy pathogens within the airflow through the air handling system. The determined electrical power can be used to identify the number and power rating of a number of sources of ultraviolet radiation (such as ultraviolet lamps) to be included in the air cleaning system.

The energy flux density required to reduce the number of pathogens in the volume of air may further be determined based on a desired percentage reduction of the number of pathogens in the volume of air. This allows the electrical power requirement to be determined for destroying a particular percentage of the pathogens in the airflow.

According to a third aspect of the present disclosure, there is provided a method of constructing an air handling system, comprising: replacing a duct of the air handling system with an air cleaning system designed according to any of the above paragraphs describing the second aspect of the present disclosure.

According to a fourth aspect of the present disclosure, there is provided a method of constructing an air handling system, comprising: replacing a duct of the air handling system with an air cleaning system according to any of the above paragraphs describing the first aspect of the present disclosure.

The methods of the third and fourth aspects allow a duct of an existing air handling system to be replaced by an air cleaning system that can destroy pathogens in the airflow.

According to a fifth aspect of the present disclosure, there is provided a method of manufacturing an air cleaning system, comprising: designing an air cleaning system according to the method of any of the paragraphs describing the second aspect of the present disclosure; and manufacturing the designed air cleaning system.

### BRIEF DESCRIPTION OF FIGURES

Specific embodiments are described below by way of example only and with reference to the accompanying drawings, in which:

FIG. 1 is a top perspective view of a ducting section of an air cleaning system.

FIG. 2 is a side view of the ducting section of FIG. 1.

FIG. 3 is a top perspective view of a casing arranged for attachment to the ducting section of FIG. 1.

FIG. 4 is a bottom perspective view of the casing of FIG. 3.

FIG. 5 is a schematic diagram of the arrangement of lamps within a casing.

FIG. 6 is a schematic diagram of a bevelled corner member of a ducting section.

FIG. 7 is a top perspective view of an air cleaning system in which the casing of FIG. 3 is attached to the ducting section of FIG. 1.

FIG. 8 is a side view of the air cleaning system of FIG. 6.

FIG. 9 is a cross-sectional view through line A-A in FIG. 7.

FIG. 10 is a nomograph providing parameter values for surface decontamination.

FIG. 11 is a schematic diagram of photons impinging on particles within a volume.

FIG. 12 is a nomograph providing parameter values for decontamination of a volume of air.

FIG. 13 is a schematic diagram illustrating reflection of a collimated beam between two surfaces.

FIG. 14 is a schematic diagram illustrating reflection of a beam within a spherical cavity.

FIG. 15 is a schematic diagram of an air cleaning system implemented in an air handling system of a building.

FIG. 16 is a flowchart of a method of designing an air cleaning system.

FIG. 17 is a method of manufacturing an air cleaning system.

### DETAILED DESCRIPTION

Implementations of the present disclosure are explained below with particular reference to air handling systems that are used in buildings. It will be appreciated, however, that the implementations described below may also be implemented in other systems that comprise an air inlet, an air outlet, and a flow of air between the inlet and the outlet. The implementations described herein may also be implemented in systems that use gases other than air.

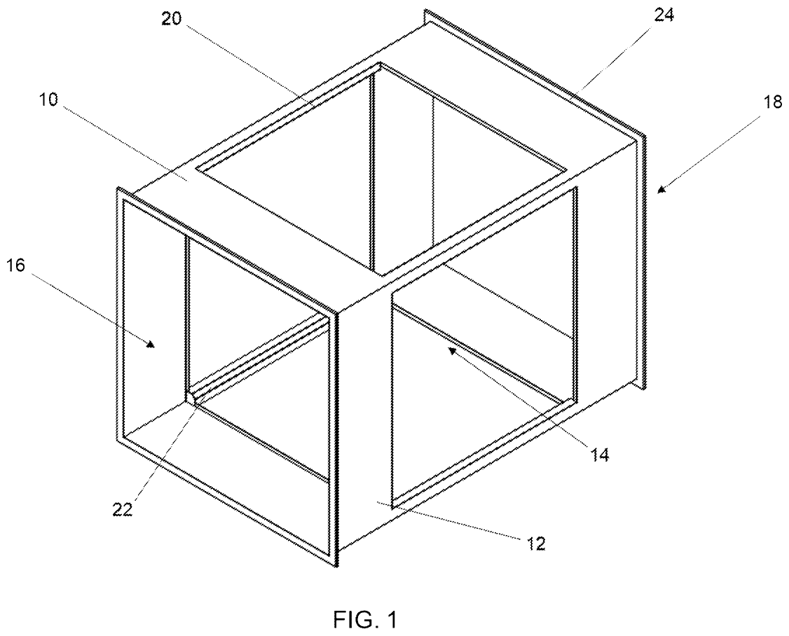

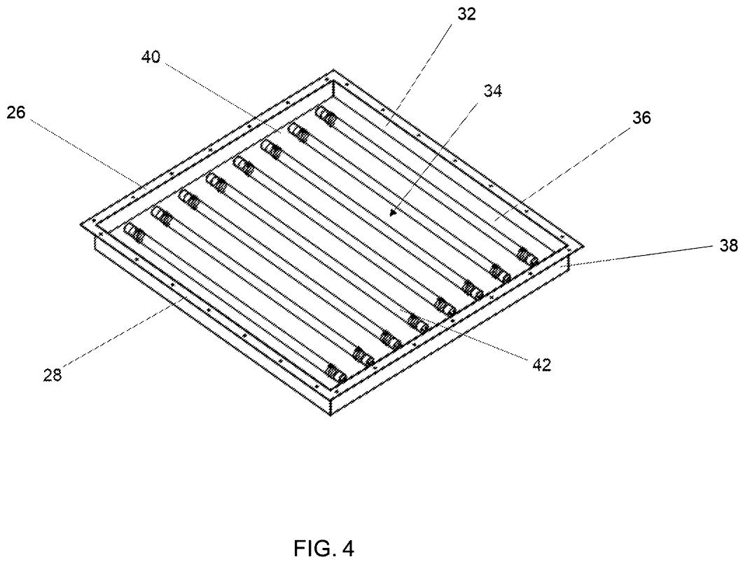

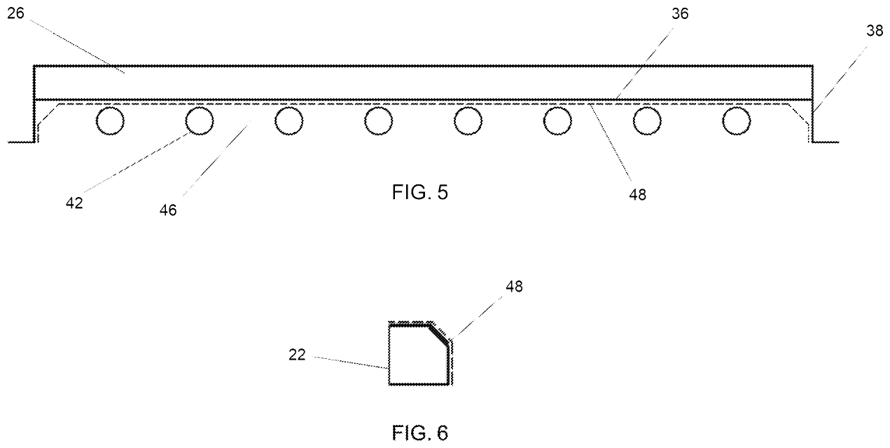

The implementations described herein reduce the circulation of contaminated air. This is achieved by providing an air cleaning system, such as the air cleaning system 50 shown in FIG. 7. The air cleaning system comprises a ducting section comprising an inlet and an outlet, such as the ducting section 10 shown in FIG. 1. The air cleaning system also comprises a source of ultraviolet radiation arranged to emit ultraviolet radiation into an interior volume of the ducting section, the interior volume being between the inlet and the outlet. The source of ultraviolet radiation may, for example, be provided in the form of one or more ultraviolet lamps 42 as shown in FIG. 4. The air cleaning system further comprises a reflective surface arranged to reflect ultraviolet radiation emitted by the source of ultraviolet radiation within the interior volume of the ducting section. The reflective surface may be capable of reflecting at least 60% of incident ultraviolet radiation. The reflective surface may, for example, be provided in the form of the layer 48 of reflective material shown schematically in FIG. 5.

In use, the ultraviolet radiation emitted by the source of ultraviolet radiation irradiates any pathogens within the airflow, thereby damaging the RNA chains of the pathogens and rendering them inactive. The reflective surface reflects the photons emitted by the source of ultraviolet radiation, thereby increasing the energy flux density within the internal volume of the ducting section. In addition, for a determined level of energy flux density to reduce the number of pathogens by a desired proportion, the increased energy flux density provided by the reflective surface can reduce the amount of electrical power needed to power the source of ultraviolet radiation.

FIG. 1 is a top perspective view of a ducting section 10 of an air cleaning system. When installed, the ducting section 10 replaces a ducting section of an existing air handling system (such as an existing HVAC system). Therefore, the dimensions of the ducting section 10 are sized to match the dimensions of the ducting section of the existing air handling system that it replaces.

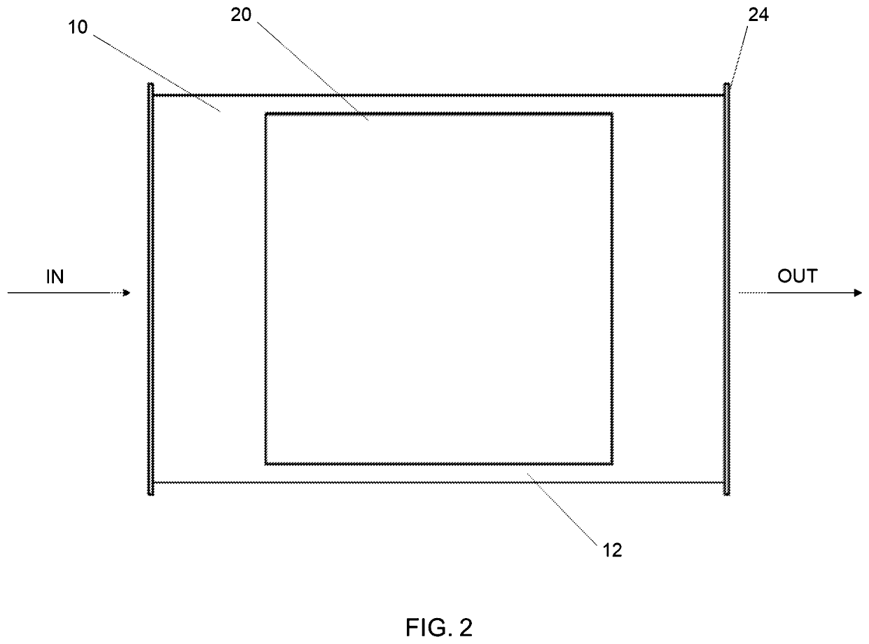

As shown in FIG. 1, the ducting section 10 comprises four walls 12 that define an interior volume 14 of the ducting section 10. As illustrated schematically in FIG. 2, the walls 12 of the ducting section 10 define an air inlet 16 and an air outlet 18. When installed, air flows into the air inlet 16 (e.g. from an upstream adjacent ducting section of the existing air handling system), through the interior volume 14 of the ducting section 10, and out through the air outlet 18 (e.g. to a downstream adjacent ducting section of the existing air handling system).

Each wall 12 comprises an opening 20, meaning that four openings 20 are shown in FIG. 1. The ducting section 10 in the example of FIG. 1 has a square cross-section. In this example, all four openings 20 are the same size, as a result of the consistent dimensions of the walls 12. The skilled person will appreciate that differently-sized openings may be implemented in ducting sections with different cross-sections.

The ducting section 10 further comprises bevelled corner members 22, which extend along the join between two perpendicular walls 12. The bevelled corner members 22 perform two functions. Firstly, they prevent pathogens from travelling along a corner region of the ducting section 10 (which may be subject to a lower level of energy flux density from the ultraviolet radiation source). For this reason, the bevelled corner members 22 are capped along their length and at each end (i.e. the inlet end and the outlet end), thereby forming a hollow member at each corner of the ducting section 10. Secondly, the bevelled corner members 22 provide structural support to the ducting section 10. FIGS. 1 and 2 show that the ducting section 10 also includes two flanges 24, which allow for attachment of the ducting section 10 to adjacent ducting sections of the existing air handling system.

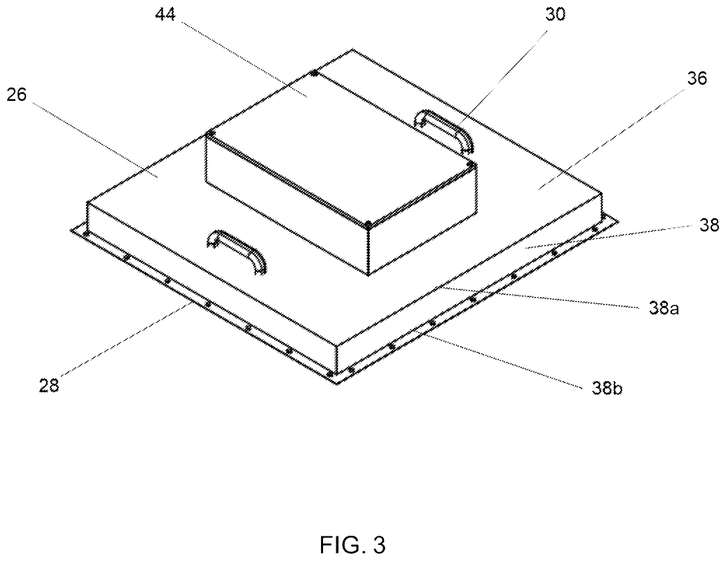

FIG. 3 shows a casing 26 arranged for removable attachment to the ducting section 10. The casing 26 in FIG. 3 is arranged to cover any one of the openings 20 of the ducting section 10 in FIG. 1. For this purpose, the casing 26 shown in FIG. 3 includes a flange 28 for attachment to an exterior surface of a wall 12 of the ducting section 10. The casing 26 also includes handles 30 that allow an operator to manoeuvre the casing 26.

As best shown in FIG. 4, the casing 26 further includes an enclosure 32. The enclosure 32 comprises an interior volume 34 defined by a back wall 36 and four side walls 38 (as best shown in FIG. 3). A first edge 38a of each side wall 38 is attached to the back wall 36. A second edge 38b of each side wall 38 is opposite to the first edge 38a. The second edges 38b of the side walls 38 define an open front face 40 of the enclosure 32.

The handles 30 are attached to an exterior side of the back wall 36 (i.e. opposite to the side of the back wall 36 that defines a surface of the interior volume 34), while the flange 28 is attached to an exterior side of the side walls 38 at the second edges 38b.

One or more sources of ultraviolet radiation (in this example, in the form of ultraviolet lamps 42) are positioned within the enclosure 32 (eight lamps 42 are shown in the example of FIG. 4). Each of the lamps 42 is positioned within the enclosure 32 of the casing 26 so as not to extend beyond a plane of the front face 40 of the enclosure 32. This ensures that the lamps 42 do not protrude into the interior volume 14 defined by the walls 12 of the ducting section 10.

As shown in FIG. 3, the casing 26 further includes an exterior enclosure 44. The exterior enclosure 44 includes power supply components (not shown) for supplying power to the lamps 42. The exterior enclosure 44 also includes circuitry for a safety cut-out switch and negative contact safety switches (not shown), which stop the power to the ultraviolet lamps 42 in the event that the casing 26 is removed from the ducting section 10. The circuitry also includes a connection to the fans that drive the airflow through an air handling system in which the air cleaning system is installed. This prevents an operator from being exposed to an airflow which is potentially contaminated with pathogens, when removing the casing 26.

Each of the ultraviolet lamps 42 is arranged to emit ultraviolet radiation. Specifically, each of the ultraviolet lamps 42 is arranged to emit UVC radiation with a wavelength of between about 180 nm and about 280 nm. Preferably, the lamps 42 are arranged to emit UVC radiation with a wavelength of at least 200 nm, in order to avoid production of ozone. A preferred range of UVC radiation is between about 210 nm and about 260 nm. This range includes wavelengths of UVC radiation that are emitted by LEDs. In particularly preferred embodiments, the lamps 42 are arranged to emit UVC radiation with a wavelength of about 222 nm or about 254 nm.

The ultraviolet lamps 42 may comprise any materials that provide for emission of UVC radiation. For example, the ultraviolet lamps 42 may be mercury fluorescent lamps or amalgam lamps. Alternatively, the ultraviolet lamps 42 may be LEDs. As a further alternative, excimer lamps and/or excimer plates may be used as, or in place of, the ultraviolet lamps 42.

FIG. 5 schematically illustrates the arrangement of lamps 42 within the casing 26. As shown in FIG. 5, the lamps 42 are evenly spaced, with gaps 46 between adjacent lamps 42. The combined area of the gaps 46 between the lamps 42 disposed in a particular casing 26 can be expressed as a percentage of the surface area of the back wall 36 of that casing 26 (i.e. an area of a surface on which the lamps 42 are disposed). The combined area of the gaps 46 between the lamps 42 disposed in a particular casing 26 may be between about 50% and about 80% of the surface area of the back wall 36 of the casing 26. In preferred implementations where a smaller number of higher-power lamps 42 are utilised, the combined area of the gaps 46 between the lamps 42 disposed in a particular casing 26 may be between about 70% and about 80% of the surface area of the back wall 36 of the casing 26, particularly preferably between about 75% and about 80% of the surface area.

When viewed from the side of the casing 26 with the lamps 42 visible, a reflective surface (in this example, in the form of a layer 48 of reflective material) is disposed behind the lamps 42. The layer 48 of reflective material therefore extends along the interior surface of the back wall 36, to provide a reflective lining within the interior volume 34 of the enclosure 32. FIG. 5 also shows that the layer 48 of reflective material extends along the interior surfaces of the side walls 38 of the enclosure 32.

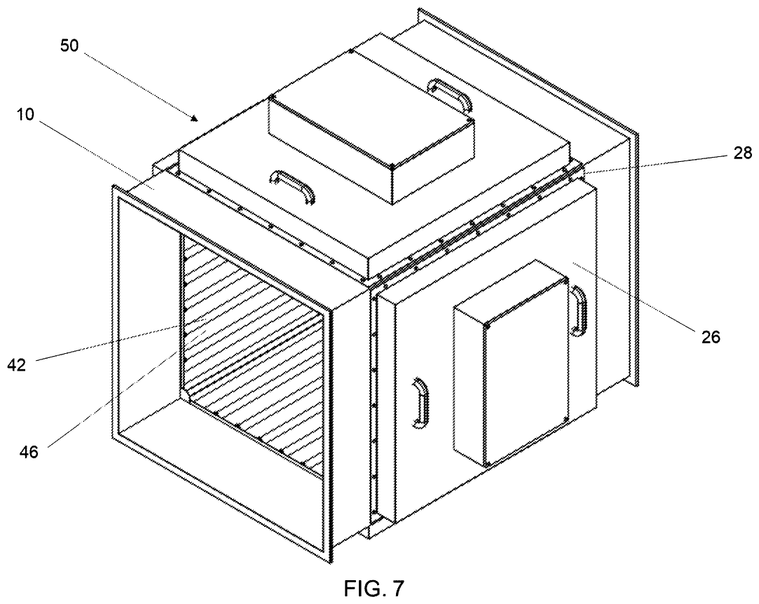

As shown in FIG. 6, the layer 48 of reflective material also extends over the surface of each bevelled corner member 22 that faces the interior volume 14 of the ducting section 10.

The layer 48 of reflective material comprises a material that is capable of reflecting ultraviolet light that is incident on the layer 48. Specifically, the layer 48 comprises a material that reflects UVC radiation without being damaged by the UVC radiation. Preferably, the layer 48 comprises a material that reflects at least 60 percent of the incident UVC radiation. More preferably, the layer 48 comprises a material that reflects at least 65 percent of the incident UVC radiation. More preferably, the layer 48 comprises a material that reflects at least 70 percent of the incident UVC radiation. More preferably, the layer 48 comprises a material that reflects at least 75 percent of the incident UVC radiation. More preferably, the layer 48 comprises a material that reflects at least 80 percent of the incident UVC radiation. More preferably, the layer 48 comprises a material that reflects at least 85 percent of the incident UVC radiation. More preferably, the layer 48 comprises a material that reflects at least 90 percent of the incident UVC radiation. More preferably, the layer 48 comprises a material that reflects at least 95 percent of the incident UVC radiation. In one example, the layer comprises polytetrafluoroethylene (PTFE), which reflects about 97 percent of incident UVC radiation. In other examples, the layer may comprise nylon, or ultra-high-molecular-weight polyethylene (UHMWPE, also known as UHMW or high-modulus polyethylene (HMPE), or any combination of these materials. The walls 12 of the ducting section 10 immediately upstream from the openings 20 and immediately downstream from the openings 20 may also be coated with a layer of material that is reflective to UVC radiation, such as PTFE.

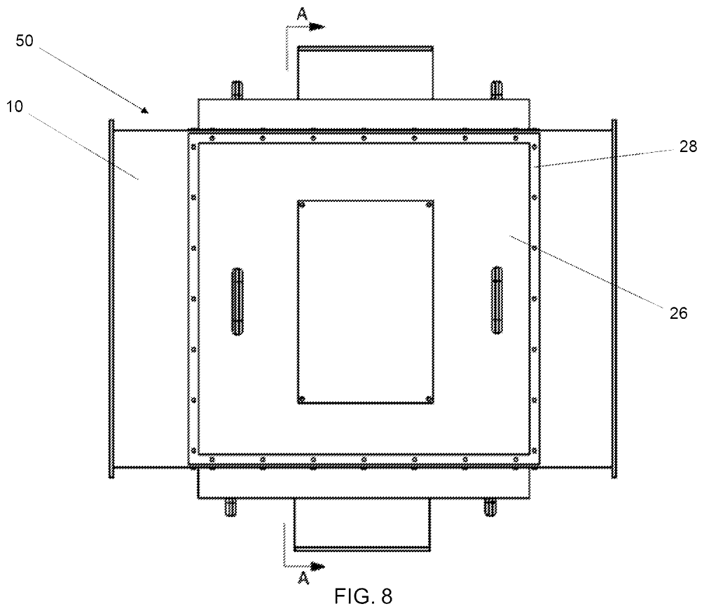

An assembled air cleaning system 50 is shown in FIG. 7. When assembled, each of the openings 12 of the ducting section 10 is covered by a casing 26. In the example shown in FIG. 7, four casings 26 are attached to the ducting section 10. Each casing 26 is attached to the ducting section 10 via screws fitted through holes in the flange 28 of the casing 26 (as seen in FIG. 7 and FIG. 8). Returning to FIG. 7, it can be seen that the casings 26 are attached to the ducting section 10 so that the longitudinal axis of the lamps 42 (i.e. along the length dimension of the lamps) is aligned with the direction of airflow through the ducting section 10 (i.e. a flow path from the air inlet 16 to the air outlet 18). In other words, the longitudinal axis of the lamps 42 is parallel to the direction of airflow through the ducting section 10.

In use, air flows into the interior volume 14 of the ducting section 10 via the air inlet 16. The ultraviolet lamps 42 emit ultraviolet radiation into the interior volume 14 of the ducting section 10. This means that the air flowing through the interior volume 14 is irradiated with ultraviolet (specifically, UVC) radiation from the ultraviolet lamps 42. The assembled air cleaning system 50 comprises four casings 26, each provided to cover an opening 20 in a respective wall 12 of the ducting section 10. This means that the ultraviolet lamps 42 emit ultraviolet radiation into the interior volume 14 from four different directions.

Emission of ultraviolet radiation from multiple different directions increases the irradiation that each virus is exposed to. For example, if ultraviolet radiation is emitted from only one direction, then the ultraviolet radiation “sees” each virus as a circular disc (i.e. as if the virus was present on a surface). Comparatively, if ultraviolet radiation is emitted from two orthogonal directions, then the ultraviolet radiation in each different direction “sees” each virus as a circular disc. However, as the emission directions are different, a greater total surface area of the virus is exposed to the ultraviolet radiation. The skilled person will, of course, appreciate that the virus surface area that is exposed to the ultraviolet radiation may also be increased by irradiating the virus from two non-orthogonal directions (e.g. opposing directions, or directions that are at an angle to one another). In addition, emission of ultraviolet radiation from multiple directions increases the likelihood of irradiating the virus in the event that the virus attaches itself to a larger particle (such as a dust particle). Comparatively, emission of ultraviolet radiation from one direction only (and assuming no reflection of radiation) could result in the larger dust particle providing a barrier to irradiation of the virus.

The layer 48 of reflective material reflects ultraviolet radiation emitted by the ultraviolet lamps 42 within the interior volume 14 of the ducting section 10. This means that the layer 48 of reflective material reflects the UVC radiation emitted by the ultraviolet lamps 42 back into the interior volume 14. In particular, the gaps 46 between the lamps 42 expose the layer 48 of reflective material to the UVC radiation emitted by the lamps 42. This allows the UVC radiation from the lamps 42 to be reflected by the layer 48 of reflective material (rather than being absorbed by the glass of the lamps 42), in order to increase the level of irradiation within the interior volume 14.

In particular, the regions in which the lamps 42 are disposed provide a lower level of reflection of the UVC radiation (around 60% reflectivity), whereas the gaps 46 between the lamps 42 provide higher levels of reflection of UVC radiation owing to the radiation being incident on the layer 48 of reflective material. This higher level of reflection may be above 60% reflectivity (as explained above), and potentially as high as over 95% reflectivity, depending on the material used in the layer 48 of reflective material. Therefore, maximising the combined area of the gaps 46 between the lamps 42 provides an increased area with higher reflectivity. The energy flux density within the air cleaning system 50 can therefore be increased by using a lower number of higher-power ultraviolet lamps 42 and maximising the gaps 46 between the lamps 42.

The energy flux density of the UVC radiation within the interior volume 14 (i.e. by the combination of the lamps 42 and the layer 48 of reflective material) is sufficient to ‘clean’ (or ‘scrub’) the air by killing any pathogens in the air flowing through the ducting section 10. The cleaned air then exits the ducting section 10 via the air outlet 18.

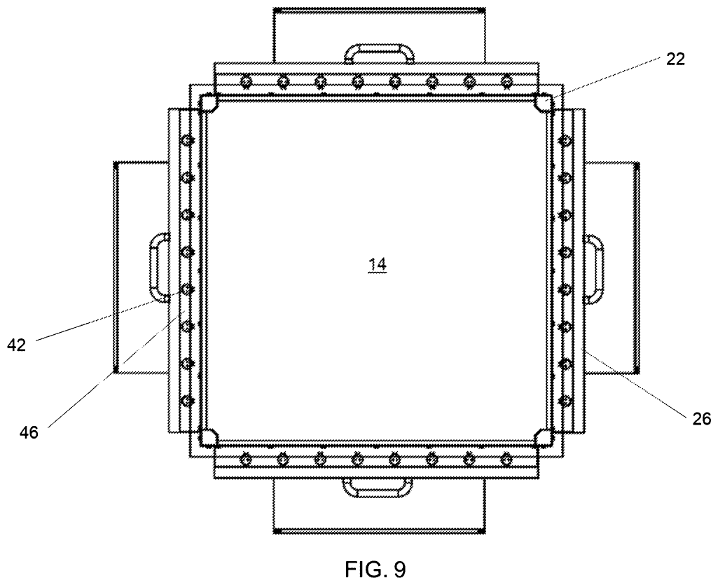

Given that the lamps 42 are disposed within the interior volume 34 of the casing enclosure 32, the lamps 42 do not protrude into the interior volume 14 of the ducting section, as best shown in FIG. 9. This mitigates any obstruction of the airflow through the ducting section 10 in use. In other words, the lamps 42 are provided in a recess so as to mitigate obstruction of airflow through the ducting section 10.

The energy flux density within the interior volume 14 of the air cleaning system 50 is sufficient to kill 99.99% of SARS-CoV-2 pathogens (i.e. the strain of coronavirus that causes COVID-19) within the air flowing through the ducting section 10. In order to kill this percentage of pathogens, the lamps 42 need to provide a particular level of energy flux density. This level of energy flux density is achieved by implementing a particular number of lamps 42 with a certain power.

The determination of the desired energy flux density (and consequently, the number and power of the lamps 42) will now be described.

The fraction of pathogens killed on a surface by a particular dose of UVC radiation follows a Poisson distribution which predicts exponential decrease with increasing dosage. This expression can be formulated as:

f=exp(−kD) (Equation 1)

Where:
f is the remaining fraction of initial pathogens (in other words, 1 minus the kill fraction);
D is the dosage of UVC radiation (often given in mJ/cm²); and
k is a constant that varies with the wavelength of the UVC radiation used and the particular pathogen in question (often expressed in cm²/mJ).

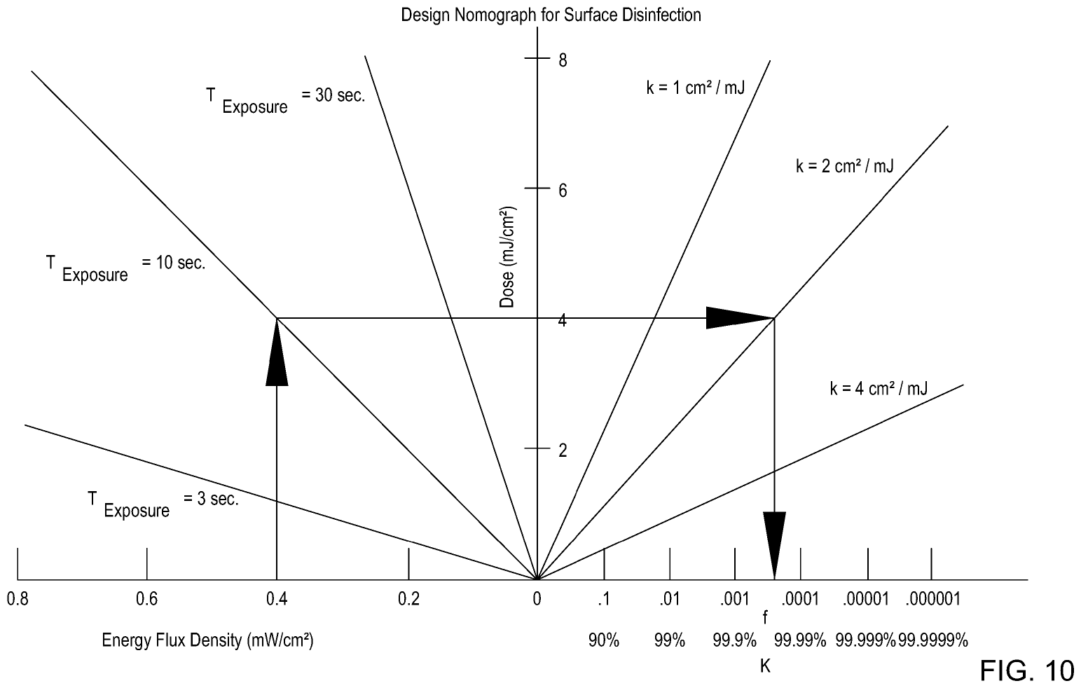

The dosage D in Equation 1 can be calculated as the energy flux density (in mW/cm²) multiplied by the exposure time. FIG. 10 is a nomograph for surface decontamination, showing the pathogen kill ratio for an energy flux density of 0.4 mW/cm² illuminating a surface for 10 seconds. The resulting dosage level is 4 mJ/cm². For a pathogen with a k value of 2 cm²/mJ, this dosage level results in a kill ratio of about 99.95%.

Equation 1 is typically used for surface irradiation. However, it can also be used in dosage calculations for airborne pathogens within a volume. This is for the reasons explained in the following paragraphs.

SARS-CoV-2 has a diameter of about 100 nm, giving it a cross-sectional area of 7.5×10⁻¹⁵ m². In an example with a duct 1 m across with an aerosol density of one particle per cubic millimetre, with one 100 nm diameter coronavirus per aerosol, the total obscured area of the photon flux will be 10⁹ particles/m 3×7.5×10⁻¹⁵ m² virus area×1 virus per aerosol×1 m path length through the air=7.5×10⁻⁵ obscuration fraction. This is 0.0075%, which is low enough to be considered negligible. This means that the beam of photons will propagate through the aerosol cloud undiminished.

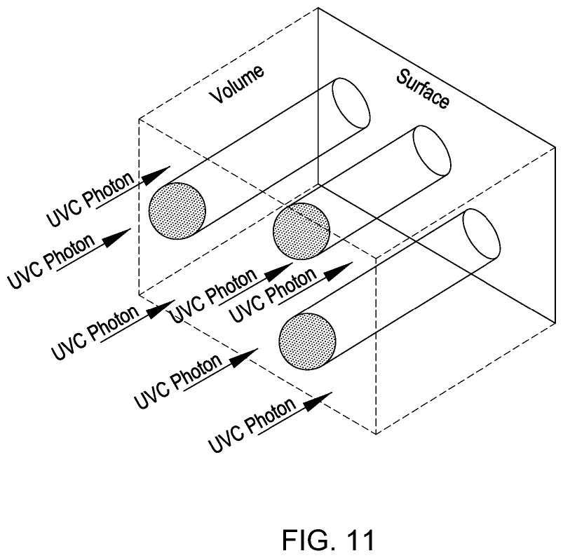

When considering a photon flux traversing virus particles suspended in an airflow, it is useful to imagine the projected area of the particles on a far wall. In light of the very low obscuration fraction (as calculated above), the number of pathogen particles in a typical airflow is far lower than the amount needed to obscure the wall completely. This means that the mortality dynamics can be computed using the exponential law given in Equation 1, but using the surface number density (given by applying f to the number density of particles on a surface—i.e. particles/m²) as the projected density of viruses in aerosols suspended in the air flow. This is illustrated schematically in FIG. 11. As shown in FIG. 11, the total obscured area of the photon beam is very small (as calculated above), meaning that the vast majority of photons are not used. Instead, they will hit the far wall.

Another consideration is the rate of attenuation of an optical beam. This is given by Beer's law, which states that a beam traversing a collection of absorbing particles of cross-sectional area A p and number density n will follow the law:

l=l₀·exp(−n·Aₚ·L) (Equation 2)

Where:
l is the flux density at a distance L into the medium; and
l₀ is the flux density at the beginning of the medium.

Equation 2 therefore determines the beam attenuation as it traverses the medium. By way of example, the number density of aerosols of 100 nm diameter necessary to attenuate a beam by 50% over a 1 m path can be calculated. The result is about 10¹⁴ particles per cubic metre, or 10⁵ particles per cubic millimetre. It is highly unlikely that particle densities this high would result from human sources (as they would appear as opaque clouds).

The quantity in parentheses in Equation 2 is often referred to as the optical depth or OD (i.e. OD=n·Aₚ·L). If OD<<1, the situation is optically thin. In optically thin situations, the photon interaction with the absorbers is weak, meaning that the beam is not very attenuated. For OD<<1, e^{-OD}≈1−OD. In other words, the fraction absorbed is just the OD. This means that the viruses that absorb photons have little effect on the beam intensity. In other words, the beam intensity is substantially constant throughout the cavity (i.e. substantially non-attenuated).

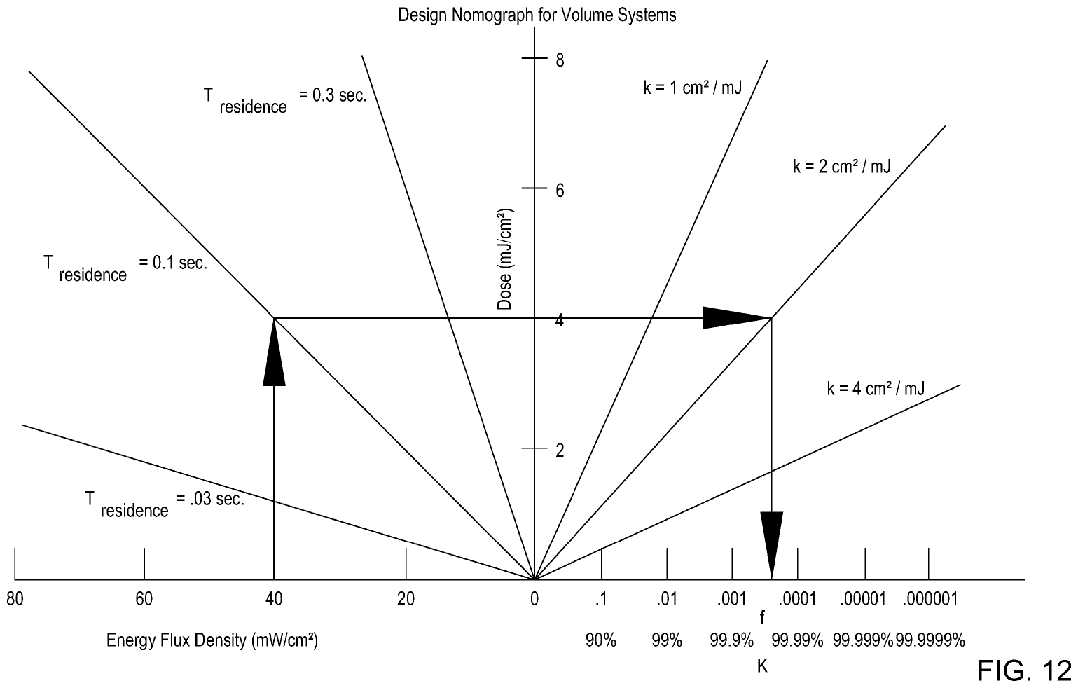

In light of the low obscuration fraction and low beam attenuation, the surface irradiation equation given in Equation 1 can be applied to irradiation of pathogens within a volume of air. For the volume case, the residence time of each particle within the air cleaning system 50 is considered, instead of the exposure time used for the surface irradiation calculation. FIG. 12 is a nomograph for volume irradiation. As shown in FIG. 12, applying an energy flux density of 40 mW/cm² to particles that are resident inside the air cleaning system 50 for 0.1 s (equivalent to a 10 m³/s airflow through a 1 m² duct) provides a dosage of 4 mJ/cm². Again, for a pathogen with a k value of 2 cm²/mJ, this dosage level results in a kill ratio of about 99.95% (as with the surface case shown in FIG. 10).

Returning now to Equation 1 and applying it to the specific case of killing SARS-CoV-2 pathogens, some values of k for known pathogens and wavelengths of UVC are given in Table 1. These values are taken from existing literature on the use of UVC for disinfection:

TABLE 1
k values for known pathogens and UVC wavelengths.

| Pathogen | Wavelength (nm) | k (cm²/mJ) |
|---|---|---|
| H1N1 | 254 | 2.2-2.9 |
| MRSA | 222 | 2.3 |
| MRSA | 254 | 0.71 |
| H1N1 | 222 | 1.8 |
| H1N1 | 222 | 2.2-2.5 |
| CoV-229E | 222 | 4.1 |
| CoV-OC43 | 222 | 5.9 |
| SARS-CoV-2 | 254 | 1.5-2.2 |
| SARS-CoV-2 | 222 | 2.2-2.3 |

From the data in the above table (in particular, the final two rows), a k value of 2 cm²/mJ can be estimated for both 222 nm and 254 nm photons for killing SARS-CoV-2 pathogens.

The total dosage, D, is the energy flux density E″ (in mW/cm²) multiplied by the residence time, t. The residence time, t, can be calculated as:

t=L/U (Equation 3)

Where:
L is the length of the internal volume (i.e. the irradiation length); and
U is the velocity of the air flow

The residence time is the time that the air spends inside the air cleaning system 50 (specifically, the time that the air spends within the section of the air cleaning system where it is exposed to ultraviolet radiation from the lamps 42). The velocities used in practical HVAC systems lie within a narrow range. For most existing systems, velocities of the order of 10 m/s are used. Some high speed systems use velocities as high as 15 m/s, but noise considerations and the costs of pumping systems keep velocities of air in ducts within this range. In cases where low noise is desired, velocities can be as little as 3 m/s. In the example calculations below, an air velocity of m/s is used.

Rearranging Equation 1 using Equation 3, we obtain an expression for the energy flux density E″:

E″=−U In(f)/(k·L) (Equation 4)

As a first example, using f=0.01 (a 99% kill ratio), k=2 cm²/mJ, U=10 m/s (as given above) and L=1 m gives E″=23 mW/cm².

For a typical duct of 1 m×1 m cross-section, the volumetric flow rate of air is 10 m³/s. If the UVC photons were emitted and used only once (i.e. not reflected), then the illuminated area would be 1 m² (height×illumination length L), and the total energy of the photons would be 230 W. Assuming a wall plug efficiency of the lamps 42 of 40%, the total electrical power required would be 575 W.

Redoing the above calculation with a desired kill ratio of 99.99% (f=0.0001) yields an energy flux density E″=46 mW/cm², requiring a total electrical power of 1151 W assuming 40% lamp efficiency. Doubling the electrical power therefore dramatically increases the kill ratio.

The above calculations also assume that UVC photons are used to irradiate the pathogens only once (i.e. they are not reflected). However, as explained in the above example, the interior of the assembled air cleaning system 50 comprises a layer 48 of reflective material such as PTFE.

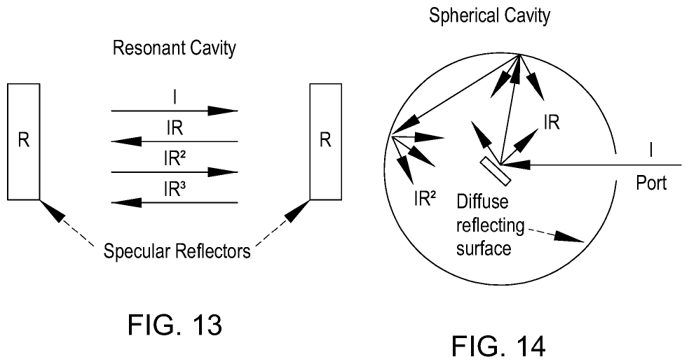

In order to determine the effect of the reflective material, a case with a perfectly collimated beam in between two perfect specular reflectors with finite reflectivity R is considered. This case is illustrated schematically in FIG. 13. On the first pass through the virus cloud, the photon flux density would be l. (The photon flux density can be converted to the energy flux density by considering the energy per photon, Eₚ=(h·c)/λ, where h is Planck's constant (6.6×10⁻³⁴ J/s), c is the speed of light (3×10⁸ m/s) and A is the wavelength of the photon in m). The return beam would have photon flux density l·R (neglecting any diffraction effects and assuming that the beam bounces back and forth between the reflectors). The second reflection would yield a beam with flux density l·R². Summing the contributions from the reflected beams would yield a total flux density lₜₒₜₐₗ=l·(1+R+R²+ . . . +Rⁿ), which simplifies using the theory of series to lₜₒₜₐₗ=/l(1−R). This means that the multiplier for the cavity with perfect reflectors is 1/(1−R). Inserting some numbers into this equation gives a factor of 10 times the flux density for a 90% reflecting wall, or a factor of 5 times the flux density for an 80% reflecting wall.

Another case that can be considered is an optical cavity in the form of an integrating sphere, illustrated schematically in FIG. 14 as a spherical cavity. Such a device can be used to measure the scattering properties of surfaces. The sphere typically has an interior coated with a very high reflectivity coating. The reflectivity is diffuse and not specular as in the case described in the above paragraph. Access ports allow a light beam to be introduced into one port and directed onto a sample inside. Reflected photons bounce around multiple times and increase the ambient photon flux density. The ratio of increase in photon flux density is given by a similar expression to above, i.e.: M=R/(1−R(1−g)), where g is the fraction of solid angle subtended by all of the ports.

The factor of R in the numerator comes from the fact that the initial beam is not counted in the calculation of the intensity field within the cavity. For the case of the integrating sphere, one of the most important factors in determining the actual multiplier is the loss of photons through the ports.

For a sphere with R=0.9 (i.e. 90% reflectivity) and g=0.33, the multiplier is 2.2 for the cavity. This is expected to be a good approximation of a cavity comprising a cube with two ends open (i.e. the geometry of the air cleaning system 50 described above).

The use of UVC lamps 42 backed by a layer 48 of reflecting material is therefore expected to lead to a significant increase in the energy flux density (and therefore photon flux density) as a consequence of the reflection of photons. PTFE is approximately 97% reflective to UVC photons. Implementing the layer of PTFE is expected to increase the energy flux density by a factor of at least two (potentially as high as three). This can further reduce the total electrical power that needs to be supplied in order to generate the energy flux density necessary to achieve the desired kill ratio. For the above example of a kill ratio of 99.99%, the total electrical power required to achieve the necessary energy flux density of 46 mW/cm² would reduce to approximately 576 W (i.e. a factor of two), even when ignoring the effects of implementing lamps that emit radiation from all four walls. When combining the effects of reflective material and lamps on all four walls, the total electrical power requirement given above will be reduced even further.

Raytracing programs can be used to compute the actual expected increase in flux density as a consequence of the reflective material, using Monte Carlo techniques.

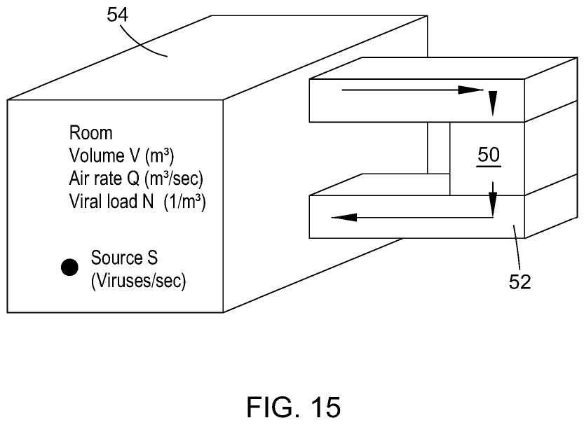

FIG. 15 is a schematic diagram of an air cleaning system (such as the air cleaning system 50 described above) implemented in an air handling system 52 of a building 54. A source of viruses that produces S viruses per second is situated in a room with volume V cubic metres. A volumetric flow rate of air of Q m³/s is removed continuously and sent to the air handling system, which includes an air cleaning system such as air cleaning system 50 described above.

The air cleaning system 50 has a kill ratio that corresponds to a fractional survival ratio f. Denoting the number of viruses per unit volume in the room in viruses/m³ allows a simple differential equation describing the rate of change of the viral load in the room to be written. This is Equation 5 below. Equation 5 states that the rate of change in the viral load is the difference between the number of viruses that are injected into the room by the source, S, minus the number of viruses that are removed:

$$\frac{{dN}_{v}}{dt} = \frac{S}{V} - \frac{{QN}_{v} ( 1 - f )}{V} \qquad ( Equation 5 )$$

The units of the rate of change of viral load are viruses per cubic metre per second.

Initially, the steady state of the system is considered. In steady state, the viral loading from the source is offset by the reduction of viruses using the air cleaning system 50. This can be investigated by setting the rate of change of viral load (i.e. the left-hand side of Equation 5) to zero. By doing this, we find that:

$${N}_{V} = \frac{S}{Q ( 1 - f )} \qquad ( Equation 6 )$$

From Equation 6, it can be determined that the difference in kill ratios is not important in maintaining a low ambient level (provided it is at least 99%), since 1−f is about 1 in either case, whether f=0.01 or 0.0001. However, increasing the amount of air through the system, Q, directly impacts the steady state level.

From a control strategy, if there was a way of measuring the viral loading, then an air handling system comprising the air cleaning system 50 could be run at higher speed (and reduced kill ratio) until the levels are lowered. The flow rate could then be lowered to better clean the air (owing to the increased residence time within the irradiation volume of the air cleaning system 50 at lower flow rate). This is an important consideration where the viral load may vary over time.

Returning to Equation 5, we now consider the decay time in the room if the pathogen source leaves. To do this, Equation 5 does not need to be solved. Instead, the terms on the right-hand side of Equation 5 that do not involve N are examined. A decay time can be defined as:

$${τ}_{Decay} = \frac{V}{Q ( 1 - f )} \qquad ( Equation 7 )$$

Substituting Equation 7 into Equation 5 and setting S=0 (i.e. no viral load) gives:

$$\frac{{dN}_{V}}{dt} = \frac{{N}_{V}}{{τ}_{Decay}} \qquad ( Equation 8 )$$

Equation 8 has the solution:

N_{V}=N_{V}⁰e^{-t/τ}^{_{Decay}} (Equation 9)

Equation 9 is a typical exponential decay. Note again that since 1−f is always about 1 regardless of whether the kill ratio is 99% or 99.99%, the decay time can simply be estimated as V/Q (which is the turnover time for the room). This is the time required by the system to cycle one room volume through itself.

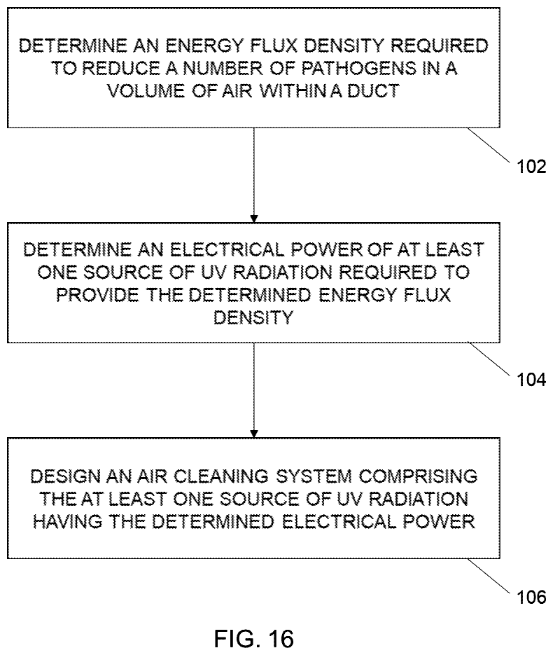

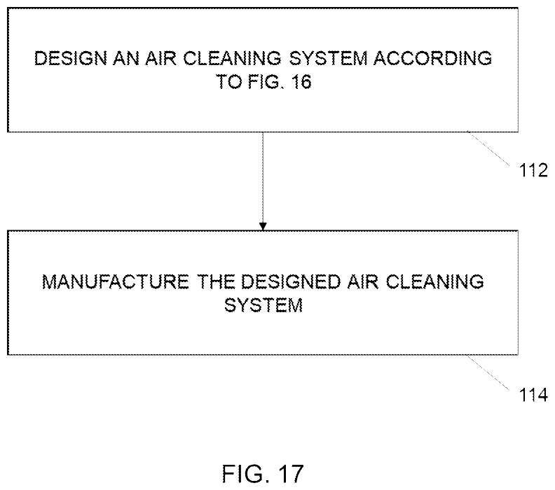

FIG. 16 is a flowchart of a method of reducing pathogens in an air handling system, such as an existing air handling system for an existing building. At 102, an energy flux density required to reduce a number of pathogens within a duct of the air handling system is determined. The energy flux density is determined based on the size of the duct and the speed of airflow through the duct. If a particular kill fraction is desired (e.g. for a building such as a hospital), then the required energy flux density may also be determined based on the desired percentage reduction of the number of pathogens in the volume of air (e.g. a 99.99% reduction). The energy flux density may, for example, be calculated using Equation 4.

At 104, an electrical power of at least one source of ultraviolet radiation required to provide the determined energy flux density is determined. The electrical power requirement may be determined based on the volumetric flow rate of air through the duct and the efficiency of the at least one source of ultraviolet radiation (e.g. the efficiency of one or more ultraviolet lamps). The electrical power requirement may further be determined based on a factor that accounts for the reflection of ultraviolet radiation.

At 106, an air cleaning system is designed. The air cleaning system comprises the at least one source of ultraviolet radiation having the determined electrical power. The air cleaning system may also comprise a plurality of sources of ultraviolet radiation that are arranged to emit ultraviolet radiation into an interior volume of a ducting section of the air cleaning system from multiple directions. In addition, the air cleaning system may comprise a reflective surface arranged to reflect ultraviolet radiation emitted by the source of ultraviolet radiation within the interior volume of the ducting section (if the electrical power determined at 104 is based on the factor that accounts for the reflection of ultraviolet radiation).

FIG. 17 is a method of manufacturing an air cleaning system. At 112, the method comprises designing an air cleaning system using the method described with reference to FIG. 16. At 114, the method comprises manufacturing the designed air cleaning system. Manufacturing the designed air cleaning system may further comprise replacing a duct of an existing air handling system with the air cleaning system manufactured at 114.

Variations or modifications to the systems and methods described herein are set out in the following paragraphs.

The air cleaning system 50 described above comprises a ducting section 10 with four walls 12. It will be appreciated that the implementations described above are also applicable to other duct cross-sections, such as rectangular, triangular or circular cross-sections. In the case of a circular cross section, the ultraviolet lamps may be provided within a casing that is arranged to cover an opening in a cylindrical surface. In this case, the casing will have an arcuate cross-section.

In the above example, the ultraviolet lamps 42 are arranged to emit ultraviolet radiation with a wavelength of 222 nm or 254 nm. It will be appreciated that other wavelengths of ultraviolet radiation may be used to reduce the number of pathogens within a volume of air. Preferably, the wavelength of UVC radiation should be high enough to avoid ozone production but low enough to effectively kill the pathogens.

In addition, the ultraviolet lamps 42 in the above example are disposed on all four sides of the duct. In alternative examples, the ultraviolet lamps may not be disposed on all four sides. In particular, the ultraviolet lamps may be disposed on only one of the sides of the duct. If one or more of the sides of the duct are free from ultraviolet lamps, those sides may be coated with a layer of reflective material in order to reflect ultraviolet radiation emitted by the ultraviolet lamps.

Although the above examples include ultraviolet lamps 42 disposed in casings 26 attached to a ducting section 10, the ultraviolet radiation may alternatively be provided by sources of ultraviolet radiation integrated within the walls of a ducting section (which may then not comprise any openings). For example, the ultraviolet radiation may be provided in the form of an ultraviolet light plate which may form part, or all, of the wall of the ducting section. Where sources of ultraviolet radiation are integrated into the walls of the ducting section, the total area of the gaps between the ultraviolet radiation sources (which expose a layer of reflective material to the ultraviolet radiation) may be expressed as a percentage of the surface area of the wall of the ducting section.

Although the above examples are described with reference to reduction of the number of SARS-CoV-2 pathogens, it will be appreciated that the above implementations may also be used to reduce the numbers of different types of pathogens within a volume of air. For example, the above implementations may be used to reduce the numbers of any of the pathogens listed in Table 1, along with influenza, MRSA and tuberculosis. The energy flux density required to reduce the numbers of a specific pathogen may be determined based on the k value for that pathogen. For pathogens with a higher k value than the k value of SARS-CoV-2, the number and power of the ultraviolet lamps may be adapted accordingly.

In order to increase the kill fraction provided by the air cleaning system 50, multiple units of the air cleaning system 50 may be provided in series.

The described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g., for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

The singular terms “a” and “an” should not be taken to mean “one and only one”. Rather, they should be taken to mean “at least one” or “one or more” unless stated otherwise. The word “comprising” and its derivatives including “comprises” and “comprise” include each of the stated features, but does not exclude the inclusion of one or more further features.

The above implementations have been described by way of example only, and the described implementations are to be considered in all respects only as illustrative and not restrictive. It will be appreciated that variations of the described implementations may be made without departing from the scope of the invention. It will also be apparent that there are many variations that have not been described, but that fall within the scope of the appended claims.

1. An air cleaning system comprising:
a ducting section comprising an inlet and an outlet;
at least one source of ultraviolet radiation arranged to emit ultraviolet radiation into an interior volume of the ducting section, the interior volume between the inlet and the outlet; and
a reflective surface arranged to reflect ultraviolet radiation emitted by the source of ultraviolet radiation within the interior volume of the ducting section, wherein the reflective surface is capable of reflecting at least 60% of incident ultraviolet radiation.

2. An air cleaning system according to claim 1, wherein the at least one source of ultraviolet radiation is provided in a recess so as to mitigate obstruction of airflow through the ducting section.

3. An air cleaning system according to claim 1, wherein the reflective surface comprises a material that is capable of reflecting at least 80% of incident ultraviolet radiation.

4. An air cleaning system according to claim 3, wherein the material is capable of reflecting at least 90% of incident ultraviolet radiation.

5. An air cleaning system according to claim 3, wherein the material comprises one or more of: polytetrafluoroethylene, PTFE, nylon, ultra-high-molecular-weight polyethylene, UHMWPE, or any combination of the foregoing materials.

6. An air cleaning system according to claim 1, further comprising a removable casing, wherein the source of ultraviolet radiation is disposed within the removable casing.

7. An air cleaning system according to claim 6, wherein the removable casing further comprises the reflective surface.

8. An air cleaning system according to claim 6, wherein the ducting section comprises at least one wall that defines the interior volume, and wherein the removable casing is arranged to cover an opening in the at least one wall.

9. An air cleaning system according to claim 6, wherein the ducting section comprises a plurality of walls that define the interior volume, and wherein the air cleaning system comprises a plurality of removable casings, each of the plurality of removable casings being arranged to cover an opening in a respective one of the plurality of walls.

10. An air cleaning system according to claim 9, wherein each of the plurality of removable casings comprises a source of ultraviolet radiation.

11. An air cleaning system according to claim 1, wherein the at least one source of ultraviolet radiation comprises a plurality of ultraviolet lamps.

12. An air cleaning system according to claim 11, wherein adjacent ones of the plurality of ultraviolet lamps are spaced apart from one another to provide a gap between the adjacent ones of the plurality of ultraviolet lamps.

13. An air cleaning system according to claim 12, wherein a portion of the reflective surface is exposed to the ultraviolet radiation through the gap between the adjacent ones of the plurality of ultraviolet lamps.

14. An air cleaning system according to claim 12, wherein the total area of the gaps is between about 50% and about 80% of an area of a surface on which the ultraviolet lamps are disposed.

15. An air cleaning system according to claim 14, wherein the total area of the gaps is between about 70% and about 80% of the area of the surface on which the ultraviolet lamps are disposed.

16. An air cleaning system according to claim 11, wherein each of the plurality of ultraviolet lamps comprises a longitudinal axis, wherein the longitudinal axis of each of the plurality of ultraviolet lamps is parallel to a flow path from the inlet to the outlet.

17. An air cleaning system according to claim 1, wherein the at least one source of ultraviolet radiation comprises a plurality of sources of ultraviolet radiation, wherein the plurality of sources of ultraviolet radiation are arranged to emit ultraviolet radiation into the interior volume from at least two directions.

18. An air cleaning system according to claim 1, wherein the at least one source of ultraviolet radiation is arranged to emit ultraviolet-C, UVC, radiation, wherein the wavelength of the UVC radiation is between about 200 nm and about 280 nm.

19. (canceled)

20. An air cleaning system according to claim 18, wherein the wavelength of the UVC radiation is about 222 nm or about 254 nm.

21-23. (canceled)

24. A method of constructing an air handling system, comprising:
replacing a duct of the air handling system with an air cleaning system according to claim 1.

25. (canceled)
